(19) United States
(12) Patent Application Publication
(10) Pub. No.: US 2002/0049405 A1
(43) Pub. Date: Apr. 25, 2002

(54) Device and method for mixing a two-part composition forming synthetic bone

(76) Inventors: Richard J. Deslauriers, Woodbury, CT (US); Robert T. Potash, S.Windsor, CT (US)

Correspondence Address: RICHARD J. DESLAURIERS, 143 WOLCOTT ROAD, WOLCOTT, CT, 06716, US

(21) Appl. No.: 09/877,171

(22) Filed: Jun. 11, 2001

Related U.S. Application Data
(60) Provisional application No. 60/241,434, filed on Oct. 19, 2000.

(51) Int. Cl. A61M 37/00; A61M 5/00

(52) U.S. Cl. 604/82

(57) ABSTRACT

A device and method for the mixing a two-part composition, where such composition may be characterized as synthetic bone being formed by the mixing of a liquid solution and a powder. The device is comprised of a syringe within a syringe, where the contents of the first syringe pass directly from the outlet of the first syringe into the second syringe. A filtering mechanism is further incorporated into the outlet of the second syringe such that the filtering mechanism restricts the removal of the powder, but allows the passage of solution. The filtering mechanism serves an innovative function that permits the application of solution to ensure saturation and then permits the removal of excess solution so as to minimize too rapid or excessive drying times of the synthetic bone. This invention further discloses a method of utilizing vacuum pressure to remove air bubbles that may be present in the synthetic bone that affects the ability of the solution and powder to form a cohesive bond.

### CROSS-REFERENCES TO RELATED APPLICATIONS

This Application is a Continuation-in-Part of U.S. Provisional Patent No. 60/241,434 with its contents incorporated herein by reference.

### STATEMENT REGARDING FEDERALLY SPONSORED RESEARCH OR DEVELOPMENT

Not Applicable

### REFERENCE TO A “MICROFICHE APPENDIX”

Not Applicable

### BACKGROUND OF THE INVENTION

1. Field of the Invention

This invention relates to the field of mixing devices, specifically those devices used in the preparation of synthetic bone being comprised of a synthetic bone powder, such as hydroxyapatite, calcium phosphate, calcium carbonate, or other similar biointegratable compounds, growth factors, and a solution, such as sodium phosphate, water, or saline, in another container. A predetermined amount of solution is mixed together with a predetermined amount of the synthetic bone powder to form a pasty composite of synthetic bone. The composition then hardens to form a material similar to natural human bone that is able to adhere to damaged bone sites, prosthetic devices, or other similar uses.

The composition begins to harden and solidify once the liquid is introduced to the powder, and therefore the mixing of the synthetic bone and the liquid must be accomplished within a specific time frame and with fastidious precision. In one scenario, the composition may consist of sporadic dry spots within the composition such that its application to the damaged bone site may not fully integrate with the damaged bone site. In another scenario where too much solution is added to the powder, the mixture becomes too saturated and therefore prevents the mixture from drying and adhering to the site in a minimal amount of time. Because the composition begins to cure immediately, and since the procedure normally occurs while the patient is being operated on, the mixing must be done accurately and quickly the first time such that the composition may be applied with minimal trauma to the patient.

2. Description of Related Art

Traditional methods of mixing provide for a predetermined amount of synthetic bone to be mixed with a predetermined amount of liquid so that the resulting composition exhibits the required properties for curing and strength that results from a certain solution to powder ratio. Forms of traditional mixing involve the use of a mortar and pestle or a bowl in conjunction with a spatula. Other methods teach the use of devices that provide a sealed chamber where the composition is mixed using an incorporated paddle or blade, or the use of a syringe for dispensing the solution in a precise manner.

These methods, though capable of achieving the proper result, are less favorable in the field because of the difficulty in ensuring the proper hydration level is achieved without over or under saturation of the powder. This drawback in using the aforementioned devices stems from their operation such that once the fluid is introduced to the synthetic bone powder, then these devices and methods do not teach a method of removing any excess solution from the synthetic bone. Therefore, a precise amount of fluid must be administered in a manner that allows for complete saturation of the synthetic bone that does not leave any dry spots or clumping, but does not over saturate the synthetic bone so that the composition takes too long to harden potentially causing further trauma to the patient or the need for a new mixture to be prepared. Furthermore, due to this drawback, it is difficult to obtain varying solution to powder ratios within the composition such that drying time can be controlled as to allow for increased or decreased drying time as specific instances may call for during its application.

The present invention discloses a mixing apparatus that seeks to address the aforementioned concerns of rapid and complete mixing of a solution and powder that begins to harden and cure immediately upon the combining of the elements. The present invention seeks to teach a method of mixing a powder and a solution, whereby rapid hydration of the powder is achieved through super saturation of a powder followed by the removal of excess solution. Additionally, disclosed herein is a mixing apparatus for use with said disclosed method of mixing that permits over-saturation of the powder ensuring complete hydration and where the proper ratios of solution to powder are achieved by the removal of the excess solution from the composition.

### BRIEF SUMMARY OF THE INVENTION

Pursuant to the present invention a method of mixing and preparing a composition of a powder and a solution is disclosed. This disclosure first teaches a method of mixing a powder and a solution to form a new composition exhibiting certain chemical properties falling within certain ranges by first super saturating said powder with said solution and the removal of excess solution once the powder is super saturated. Further disclosed is an invention that utilizes two chambers, characterized as a mixing chamber with filtration system and a solution chamber in order to complete the method of mixing as herein described.

According to this invention, a method of mixing a composition being comprised primarily of a powder, a solution, and growth factors is disclosed and taught.

According to the preferred embodiment, a solution housed in a first chamber is transferred into a second chamber, which second chamber is holding a powder, and such that there is more solution being passed into the second chamber than that is required to attain the proper ratio of solution to powder for the composition eliciting the requisite curing time for the resulting composition. The solution to powder ratio provided for pursuant to this disclosed method of mixing therefore exceeds the ratio actually required in order for the composition to cure properly after mixing is completed during its application. The solution and powder are then mixed by manually shaking the chamber to form a milky substance. The excess solution is then removed from the mixing chamber through the use of an integrated filter system, whereby the filter prevents the saturated synthetic bone particles from leaving the mixing chamber, but allows excess clean solution to pass back into the solution chamber to be expelled as waste.

According to this disclosed method of mixing, a mixture of solution and powder remain in the chamber such that varying levels of saturation is permitted, whereas the resulting composition can be left wet for slow drying, moist for standard drying time, or dry for faster drying time.

According to another embodiment of the disclosed invention, the mixing device facilitates the removal of harmful air bubbles and other gases from the final composition forming a synthetic bone, where such air bubbles affect the two substances ability to form a cohesive bond. To facilitate the removal of air bubbles within the mixing chamber a vacuum may be applied to the mixing chamber thereby quickly and more completely removing any air and gas particles that may be present in the composition forming synthetic bone.

According to this disclosed invention, the chambers are constructed of an inexpensive material, such as plastic, and whereas the chamber may not be broken or damaged easily during storage or during its use. Additionally, the material used should be of negligible surface tension such that compositions that are mixed within the chamber do not adhere to the sides of the chamber, thereby facilitating the removal of the composition from the disclosed device.

According to the preferred embodiments of this invention, the passage of the clean solution is permitted without any particles of the saturated powder passing through the chamber pursuant to an integrated filter. In the preferred embodiment, the filter has a porosity measurement being no greater than 100 microns, but small enough to retain any particles of the powder from passing through said filter. Thereby resulting in all the solution not required to achieve the proper curing of the resulting composition to be removed from the chamber.

According to the preferred embodiments of this invention, the chambers are constructed with the characteristics of a syringe, and including characteristics as having a plunger mechanism to activate the first and second chambers, and whereby the tips of the first and second syringe are manufactured as to permit the attachment of the first to the second.

According to another preferred embodiment of this mixing device, the plungers have an integrated mechanism to lock the plunger at certain predetermined positions within their respective chambers.

According to another embodiment of this mixing device, the plunger and chambers are threaded, such that the plunger is activated through the twisting and screwing of the plunger into the chamber, and which causes pressure to be either applied or removed from the chambers.

According to another preferred embodiment of this disclosed mixing device, the one or both of the chambers are clear. Whereby, pursuant to the clear chambers, the user is able to view the contents inside of the individual chambers. Additionally, the chambers may be marked such the user is able to select higher or lower saturation levels for both the mixture and the resulting composition.

According to another preferred embodiment, the plunger of the second, or mixing, chamber has a means for agitating the mixture within the second chamber incorporated into the tip of the plunger, whereby the activation of the plunger either through twisting the plunger within the second chamber, or other means accomplishing the same agitation of the mixture.

### BRIEF DESCRIPTION OF THE DRAWINGS

FIG. 1(a) depicts a schematic side-view of the first chamber.

FIG. 1(b) depicts a side view schematic of the mixing chamber

FIG. 1(c) depicts an exploded view of the filter cap.

FIG. 2(a) depicts a side-view of the first chamber being attached to the mixing chamber.

FIG. 2(b) depicts a schematic side view of the first chamber's contents being mixed with the contents of the mixing chamber.

FIG. 2(c) depicts a schematic side view of the excess solution being transferred to the first chamber.

FIG. 3 depicts the resulting composition being removed from the mixing chamber.

### DETAILED DESCRIPTION OF THE INVENTION

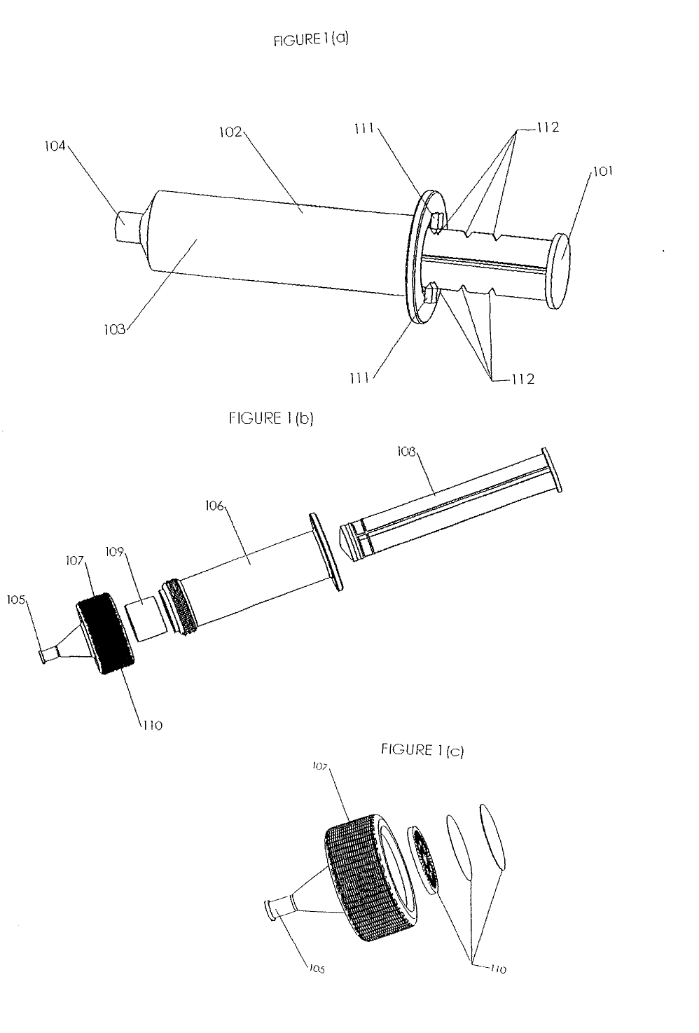

FIGS. 1(a)(b)(c) show the preferred embodiments of the disclosed device, as described herein, being comprised of three primary parts, Solution Chamber 102 and Mixing Chamber 106, and Filter Cup 107. Both Solution Chamber 102 and Mixing Chamber 106 are constructed of a molded plastic resin, whereas the inner surface of the Mixing Chamber 106 exhibits negligible surface tension. Furthermore, both Solution Chamber 102 and Mixing Chamber 106 are designed in the shape of syringe and additionally, Solution Chamber 102 having Solution Plunger 101 and whereas, Mixing Chamber 106 having Mixing Plunger 108 and Filter Cup 107.

Specifically, Solution Chamber 102 is shown such that the device is manufactured in the shape of a syringe and with Solution Plunger 101 being inserted into Solution Chamber 102. Solution Plunger 101 provides for Locking Points 112 and whereby the Solution Chamber 102 has Locking Member 111, such that Locking Points 112 provide a means of securing Solution Plunger at certain predetermined positions such that once Locking Member 111 reaches a Locking Point 112 additional force is required to disengage Locking Member 111 from Locking Points 112. Solution 103, packaged within Solution Chamber 102 is a predetermined amount of solution such as sodium phosphate, saline, a combination of saline and sodium phosphate, or other similar solution, and, also, whereby certain growth factors may further be integrated into said solution. The amount of Solution 103 being included is an amount to ensure over saturation of Powder 109, shown in FIG. 1(b). Further shown in FIG. 1(a), Connecting Tip 104 located at the end of Solution Chamber 102, whereby Connecting Tip 104 serves as a method of connecting Solution Chamber 102 to Mixing Chamber 106, shown in FIG. 1(b), through Cup Tip 105 located on Filter Cup 107 of Mixing Chamber 106, as shown in FIG. 1(b).

Referring to FIG. 1(b), Mixing Chamber 106 manufactured in the shape of a syringe and having parts Filter Cup 107 and Mixing Plunger 108 is depicted. The inner surface of Mixing Chamber 106 has a negligible surface tension so as to prevent any Synthetic Powder 109 from adhering to the inner surface of Mixing Chamber 106. The Filter Cup 107 is manufactured as to allow the attachment and removal of Filter Cup 107 from Mixing Chamber 106 when the user desires to remove the contents of Mixing Chamber 106, whereas said means of attachment is accomplished through the use of a threaded inner surface of Filter Cup 107 and a threaded outer surface of Mixing Chamber 106.

Furthermore in referring to an exploded view of Filter Cup 107 in FIG. 1(c), Filter Cup 107 is shown being manufactured to include Filter System 110. Whereas Filter System 110, including Filter 114 that only permits Solution 103 to pass and whereas Synthetic Powder 109 is prevented from passing through Filter System 110.

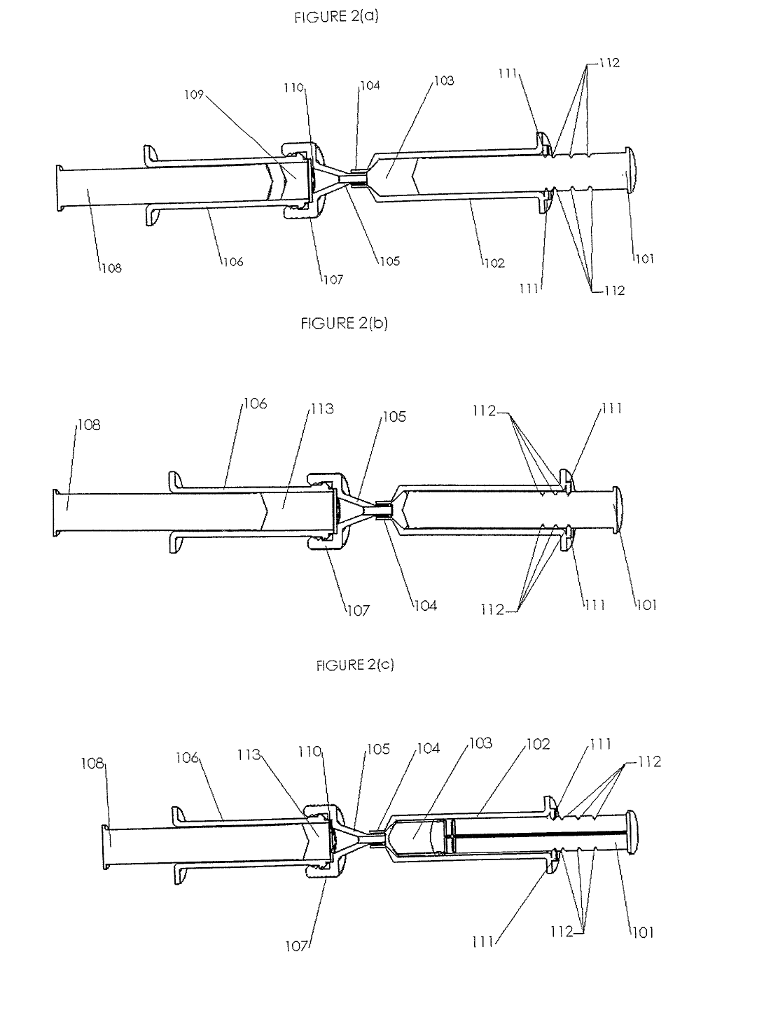

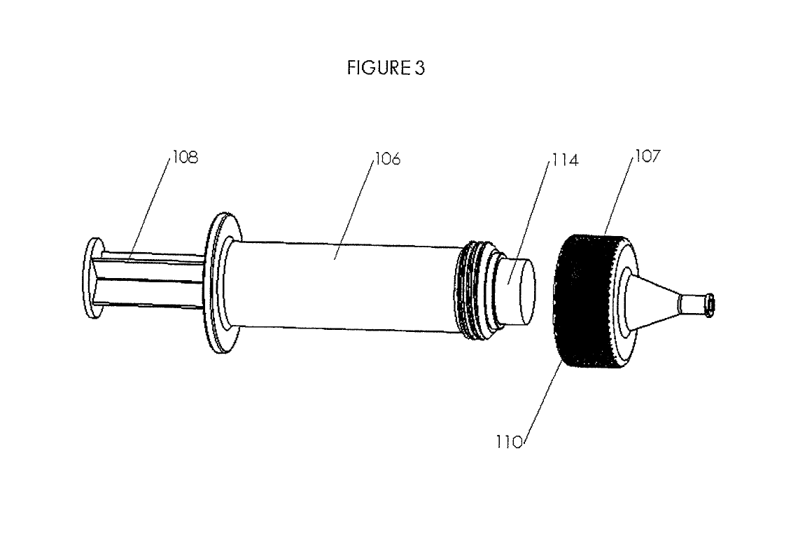

FIGS. 2(a)(b)(c) depict a side view schematics of how the disclosed mixing device, referred to above, operates pursuant to the forthcoming disclosed method of mixing a two-part composition. Super saturation of Synthetic Powder 109 is accomplished by introducing an excess amount of Solution 103 than that is actually required in eliciting the necessary solution to powder ratio for an effective application of the resulting composition of Synthetic Bone 114 as shown in FIG. 3.

FIG. 2(a) shows Solution Chamber 102 filled with an amount of Solution 103 exceeding the amount necessary to be mixed with a predetermined amount of Synthetic Powder 109 contained in Mixing Chamber 106 in order to produce Synthetic Bone 113, shown in FIG. 3, at a desired solution to powder ratio which elicits a specific duration of time for both curing and drying of the resulting Synthetic Bone 113. Mixing Chamber 106 is shown having a predetermined amount of Synthetic Powder 109, which is to be exposed to Solution 103 in the course of completing this disclosed method of mixing. FIG. 2(a) shows the device, disclosed above in reference to FIGS. 1(a)(b)(c), whereby Solution Chamber 102 is connected to Mixing Chamber 106 through the insertion of Connecting Tip 104, at the end of Solution Chamber 102, into Cup Tip 105.

FIG. 2(b) depicts the device, disclosed herein, operating pursuant to the principles of the method of mixing as herein described. After attaching the Solution Chamber 102 and Mixing Chamber 106 described above, the disclosed device operates as a single unit. Whereas, the activation of Solution Plunger 101 injects Solution 103 through Connecting Tip 104 and Cup Tip 107 and into Mixing Chamber 106 wherein Synthetic Powder 109 is housed. When Solution Plunger 101 is fully depressed leaving no Solution 103 in Solution Chamber 102, Solution 103 and Synthetic Powder 109 are combined and mixed to form Supersaturated Composite 113, accomplished most efficiently by manually shaking the device, such that clumping and dry spots within Supersaturated Composite 113 are quickly and efficiently eliminated, which results are accomplished through this disclosed method mixing through super saturation.

FIG. 2(c) depicts the manner in which excess Solution 103 is removed from Mixing Chamber 106 as described in this disclosed method of mixing involving super saturation. Excess Solution 103 is removed from Supersaturated Composite 113 and Mixing Chamber 106 in order to attain a desired solution to powder ratio that provides for a specific duration of time that is required for Synthetic Bone 114 to cure and harden. Excess Solution 103 is removed through the drawing back on Solution Plunger 101 until Locking Member 111 engages with one of Locking Points 112. Upon the drawing back on Solution Plunger 101, a vacuum is created in Solution Chamber 102 such that any excess Solution 103 is pulled back through Filter System 110, which results in forming Synthetic Bone 114 within Mixing Chamber 106. This process allows only the excess solution 103 to be removed, thereby leaving only the required amount of Solution 103 to thoroughly combine Synthetic Powder 109 with no dry spots or clumping. By increasing the draw on Solution Plunger past said first Locking Point 111, thereby increasing the vacuum pressure applied to Mixing Chamber 106 and drawing additional Solution 103, the ratio of solution to powder is lowered that results in a reduced duration of time needed for Synthetic Bone 114 to dry.

Shown in FIG. 3 is the method in which the resulting composition of Synthetic Bone 114 is easily removed from the Mixing Chamber 106. Filter System 110 and Filter Cup 107 are designed to allow simplified removal of them from the tip of mixing chamber 106, whereby upon the removal of filter 107 and cap 108 allows the composition to be quickly removed from mixing chamber 106. Once Filter Cup 107 and Filter System 110 are removed, then the activation of Mixing Plunger 108 is used to facilitate removing Synthetic Bone 114 from Mixing Chamber 106. The resulting Synthetic Bone 114 is at the ratio of solution to powder, which ratio correlates to the most efficient level for the user's intended time needed for Synthetic Bone 114 to harden and set in for the specific application, which may be needed.

What is claimed:

1. A device that provides the mixing of a first and second substance, and the ability to remove an amount of a first substance through a filter while retaining the mixture, or composition, and the second substance, whereby such device is comprised of:
Two separate elongated chambers, whereas the first chamber holding an amount of a first substance and the second chamber holding an amount of a second substance, and whereby each chamber has a corresponding plunger for either adding or removing pressure from the chambers in transferring the substances from one chamber to the other; and
Where the second chamber incorporates a filter within the tip of the second chamber, which filters has a porosity no greater than the smallest particle size of the substance to be retained, and
Whereby the tip of the first chamber incorporates a method of forming an airtight seal upon the first chamber being attached to tip of the second chamber also having a means of attachment to the tip of the first chamber.

2. The device as disclosed in claim 1, whereby the filter and tip of the second chamber is then removable from said first chamber to facilitate the removal of the resulting composition.

3. The device as disclosed in claim 1, whereby one or both of the chambers are clear so as to permit visualization of both the mixing and the water concentration.

4. The device as disclosed in claim 1, whereby the device allows a vacuum to be applied to the second chamber for degassing, or the removal of air, from the second chamber, which process maximizes the wetting of the composition.

5. The device as disclosed in claim 1, whereas the plungers for one or both of said chambers have a means of being secured in one or more positions whereby the positions allow the device to add or remove a specific amount of one or both of said substances being transferred.

6. The device as disclosed in claim 1, whereas the plungers for one or both of said chambers is threaded and the corresponding chamber is threaded to enable additional actuation forces to be applied to the chambers pursuant to the plunger being further screwed into the chamber.

7. The device as disclosed in claim 1, whereas the plunger for the mixing chamber acts as a mixing unit.

8. The device as disclosed in claim 1, whereas the tip of mixing chamber is adaptable to an external application device such as a catheter tube, made of rubber, plastic, metal, or composition thereof, and which application device in connection with compression fractures.

9. The device as disclosed in claim 1, whereas the filter used in no greater than 100 microns

10. A method of mixing a two-part composite through the process of super saturation and release as disclosed in the following process;
Whereby a first substance being a solution is combined with a second substance being a powder that results in a third material being a composite, whereas such composite is of a specific solution to powder ratio that correlates to a specific time that is required for said composite to set or harden;
Where the solution is transferred from the first chamber into the second chamber having a predetermined amount of powder, whereas the amount of solution being contained in the first chamber is of an amount of solution that exceeds the required solution to powder ratio necessary to elicit the requisite time for said resulting composite to cure and harden; and
Whereby the composite is used to mix the solution and powder to form the resulting composite through manually shaking said second chamber until the powder is now super saturated being entirely moist and suspended in the aqueous solution such that there is no clumping or dry portions of the powder within said resulting composite; and
Whereby the super saturated composite is released of the excess solution by passing the composite through a filter, where said filter retains all of the saturated powder particles and permits only the excess solution to pass through, and wherefrom the implementation of this process results in a moist composite having no dry spots or clumping with said composite.

11. The method as disclosed in claim 10, whereby through the application of vacuum pressure to the second chamber the composition is degassed removing excess gas molecules suspended in the composite chamber and simultaneously released of any excess soultion, whereby the resulting composite is in the desired solution to powder ratio.

12. The method as claimed in claim 10, wherein the solution is a mixture of saline and sodium phosphate.

13. The method as claimed in claim 10, wherein the solution is any solution capable of forming a chemical bond with the powder, or second substance.

14. The method as claimed in claim 10, wherein the solution contains growth factors that serve to augment the integration of the composition with the natural bone site.

15. The method as claimed in claim 10, wherein the powdery substance is a synthetic, such as hydroxyapetite, calcium phosphate, or any other similar synthetic bone powder.

16. The method as claimed in claim 10, wherein the powdery substance is any powder that requires the combination with a solution for activation.

17. The method as claimed in claim 10, whereby the solution to powder ratio, or wetness, of the resulting composite so produced is selectable by removing different volumes of solution from the chamber.

18. The method as claimed in claim 10, whereby the temperature of the solution or resulting composition is raised to approximate body temperature and which warming serves to facilitate crystallization and curing of the resulting composition.

19. The method as claimed in claim 10, whereby the moisture level of the resulting composite produced can be varied, such as to produce minimal solution to powder ratio or a maximum solution to powder ratio.

20. The method as disclosed in claim 10, whereby the resulting composition produced is degassed and completely devoid of any air particles.
